(19) United States
(12) Patent Application Publication
(10) Pub. No.: US 2007/0141616 A1
(43) Pub. Date: Jun. 21, 2007

(54) Microreactor

(75) Inventor: Yukio Sudo, Minami-ashigara-shi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/657,538

(22) Filed: Jan. 25, 2007

Related U.S. Application Data
(62) Division of application No. 10/673,488, filed on Sep. 30, 2003.

(30) Foreign Application Priority Data
Sep. 30, 2002 (JP) 285454/2002

(51) Int. Cl. C12Q 1/68; G01N 33/53; C12M 1/34

(52) U.S. Cl. 435/6; 435/7.5; 435/287.2

(57) ABSTRACT

The present invention provides a reactive microreactor wherein a group of the following formula (1) which can bind to a member of specifically binding partner, is bound to a part or entirety of a wall surface of a channel:
-L-SO₂—X¹ (1)
wherein X¹ represents —CR¹═CR²R³ or —CHR¹—CR²R³Y; R¹, R² and R³ independently represent an atom or a group selected from the group consisting of a hydrogen atom, a C1-6 alkyl group, a C6-20 aryl group, and a C7-20 aralkyl group having a C1-6 alkyl chain; Y represents a group which can be substitutable by a nucleophilic reagent or a group which is released as “HY” by a base; and L represents a linking group. The microreactor according to the present invention has advantages that it requires a less amount of a reagent and achieves a higher S/N ratio.

### FIELD OF THE INVENTION

The present invention relates to a biological material bound microreactor, in which the biological material such as DNA and protein, which is very useful for analyses of expression, mutation and polymorphism of a gene and proteomics analysis, is fixed on a wall surface of a channel of a microreactor.

### BACKGROUND ART

Technology is rapidly developing to analyze effectively a gene function in various living organisms. In order to analyze a nucleotide sequence of a DNA or a DNA fragment, a technique is being attempted wherein many DNA fragments or nucleotide derivatives such as synthetic oligonucleotides are fixed to a wall surface of so-called microreactor and the nucleotide sequence of the DNA or the DNA fragment is analyzed.

As a method for simultaneous detection of various types of DNAs (or mRNAs,) a DNA chip has been developed. In the DNA chip, the DNA is fixed on a plate such as a glass plate. The DNA as a test sample is to be developed on this plate and to be subjected to hybridization. Therefore, it is necessary to dilute the test sample to such an amount of a liquid that an entire surface of the DNA chip is covered. For example, a normal Stanford type DNA microarray requires at least 50 μl of the amount of a liquid of the test sample. In addition, the DNA as the test sample developed on the plate diffuses slowly, and hence, the test sample DNA involving in a reaction is a few percent. Due to such problem, the so-called DNA chip developed on the plate has a defect in that a desirable detection sensitivity is not obtained. On the other hand, a microreactor system allows concentrating the test sample to a few μl and contacting the test sample with all the fixed DNAs in order by flowing the liquid. Therefore, it is possible to design a more sensitive detection system than the DNA chip on the plate.

The microreactor is being used not only for the detection of the DNA as mentioned above, but also for the detection of protein, antigen, antibody, receptor, ligand and RNA. In the microreactor in such system, a material which specifically binds to the test sample should be fixed.

In order to commercialize so-called biological material bound microreactor in which a specific binding material is fixed, a technique for stably binding the specific binding material such as many DNA fragments, oligonucleotides, antigens or antibodys on the wall surface of the channel inside the microreactor, is required.

The following method for binding and fixing DNA fragment or the oligonucleotide, which is previously prepared, on the surface of a solid phase carrier has been known depending on the types of DNA fragments and the types of solid phase carriers.

(1) In the case where the DNA fragment to be fixed is a cDNA (complementary DNA which was synthesized using an mRNA as a template) or a PCR product (the DNA fragment prepared by amplifying a cDNA by PCR method), it is general that cDNA or PCR product is spotted on the surface of a glass pieces which was surface-treated with a polycationic compound (e.g., polylysine, and polyethylene imine) by a spotter device equipped in a DNA chip preparation apparatus, and is electrostatically bound with the glass surface using an electric charge of the DNA fragment. A method for treating the glass surface includes the method using a silane-coupling agent having an amino group, an aldehyde group, an epoxy group or the like. In the surface treatment using the silane-coupling agent, an amino group or an aldehyde group is fixed on the glass surface by a covalent bond. Therefore, in comparison with the case of the surface treatment with the polycationic compound, the group is fixed more stably on the glass surface.

As a modified method for using the electric charge of the DNA fragment as described above, there is reported a method wherein a PCR product modified with an amino group is suspended in SSC (standard salt-citric acid buffer solution), contacted to the surface of a sililated slide glass and is incubated, and then a treatment with sodium borohydride and a heating treatment are carried out subsequently. However, there is a problem that this fixing method does not always enable yielding an enough fixing stability of the DNA fragment.

(2) In the case where an oligonucleotide to be fixed (probe molecule) is a synthetic oligonucleotide, a method is also known in which an oligonucleotide to which a reaction activating group is introduced, is synthesized, the oligonucleotide is contacted to the surface of a solid phase support subjected previously to surface treatment to form the reactive group, and the oligonucleotide is bound and fixed to the surface of the solid phase support by the covalent bond. For example, there are known methods that a solid phase support, in which an amino acid group is introduced to its surface, is reacted with the oligonucleotide, to which an amino acid group is introduced, in the presence of PDC (p-phenylene diisothiocyanate), and that the slide glass is reacted with the oligonucleotide to which an aldehyde group is introduced. These two methods have an advantage that the oligonucleotide is bound and fixed stably to the surface of the solid phase support, in comparison with the method for fixation by static binding using electric charges of the DNA fragment as described in (1). However, in the method under the presence of PDC, the reaction of PDC with the oligonucleotide to which the amino acid group is introduced is slow. In the method using an oligonucleotide to which an aldehyde group is introduced, the stability of Schiff base which is a reaction product, is low and therefore hydrolysis easily occurs. These are problems.

On the other hand, genome analysis has been almost completed and “proteome and proteomics studies” are being preceded to provide essential information for learning finally a sense of gene information and simulating a life activity of a cell. The proteome means an entire set of proteins which are translated and produced in a specific cell, tissue and organ. In addition, research fields of a higher degree information analysis of a chemical structure, a total amount, a period of expression, post-translation modification, aggregation formation and the like is called “proteomics.”

Proteome study includes profiling of a protein, identification and precision analysis of the protein, analysis of interaction networks, construction of proteome database, and application of results of these analyses to the life science study.

Of these analyses, methods for the analysis of interaction networks include yeast two-hybrid method, phage display method, methods using affinity capture including immune precipitation method and BIA-MS method, and column switching-mass analysis method (Method for analyzing proteome, 163-211, Youdosya, 2000). All the methods for the analysis of interaction as described above has not achieved a high throughput analysis.

Schreiber et al. reported a protein microarray method for the high throughput analysis of interaction of proteins (Science, 289, 1760-1763, 2000). In this method, an aqueous solution of protein is spotted on the slide glass having an aldehyde group, followed by blocking with BSA solution, and then, a protein solution is reacted and detection is carried out by a fluorescent scanner. This method has a problem that the stability of Schiff base which is a reaction product of the aldehyde group and the amino group is low and therefore hydrolysis easily occurs.

Japanese Patent Publication No. 1995-53108 discloses a method for fixing the protein on a solid phase by introducing a hydrophobic polypeptide to a terminal of the protein.

Japanese patent No. 2922040 discloses a method for fixing an antibody protein by a protein A molecular membrane.

### DISCLOSURE OF THE INVENTION

An object to be solved by the present invention is to provide a reactive microreactor capable of rapid and stable biding and fixation; a biological material-bound microreactor wherein at least one of a member of specifically binding partner is bound and fixed to a wall surface of a channel inside the reactive microreactor, and the method for production thereof. Another object to be solved by the present invention is to provide a method for detecting a target substance which is another member of the specifically binding partner by using the biological material-bound microreactor mentioned above.

As a result of an intensive study by the inventors for solving the objects described above, they produced a biological material-bound microreactor wherein at least one of a member of specifically binding partner is bound to the wall surface of the channel inside the reactive microreactor by a covalent bond via a sulfonyl group. As the result, they found that the member of specifically binding partner can be rapidly and stably bound to the wall surface of the channel inside the microreactor, and the target substance can be efficiently and sensitively detected. The invention has been completed on the basis of these findings.

The invention provides a reactive microreactor wherein a group of the following formula (1) which can bind to a member of specifically binding partner, is bound to a part or entirety of a wall surface of a channel:
-L-SO₂—X¹ (1)
wherein X¹ represents —CR¹═CR²R³ or —CHR¹—CR²R³Y; R¹, R² and R³ independently represent an atom or a group selected from the group consisting of a hydrogen atom, a C1-6 alkyl group, a C6-20 aryl group, and a C7-20 aralkyl group having a C1-6 alkyl chain; Y represents a group which can be substitutable by a nucleophilic reagent or a group which is released as “HY” by a base; and L represents a linking group.

Another aspect of the present invention is to provide a method for producing a reactive microreactor, which comprises contacting a microreactor where a reactive group is introduced on the surface with a disulfon compound of the following formula (2):
X¹—SO₂-L²-SO₂—X² (2)
wherein X¹ and X² independently represent —CR¹═CR²R³ or —CHR¹—CR²R³Y; R¹, R² and R³ independently represent an atom or a group selected from the group consisting of a hydrogen atom, a C1-6 alkyl group, a C6-20 aryl group and a C7-26 aralkyl group having a C1-6 alkyl chain; Y represents a group which can be substituted by a nucleophilic reagent or a group which is released as “HY” by a base; and L² represents a linking group.

Further aspect of the present invention provides a biological material-bound microreactor, wherein a group of the following formula (3) having a residual group of a member of specifically binding partner is bound to a part or entirety of a wall surface of a channel:
-L-SO₂—X-A (3)
wherein L represents a linking group which bind —SO₂—X-A to the wall surface of the channel inside the microreactor; X represents —CR¹¹(R¹²)—CR¹³(R¹⁴)—; R¹¹, R¹², R¹³ and R¹⁴ independently represent a hydrogen atom, a C1-6 alkyl group, a C6-20 aryl group or a C7-26 aralkyl group having a C1-6 alkyl chain; A represents a residual group of the member of the specifically binding partner.

Further aspect of the present invention provides a method for producing the biological material-bound microreactor, which comprises a step of contacting the reactive microreactor according to the present invention with at least one of a member of specifically binding partner having a reactive group which reacts with a group of the aforementioned formula (1) and forms a covalent bond.

Further aspect of the invention provides a method for detecting a target substance, which comprises steps of: contacting the biological material-bound microreactor according to the invention with a sample containing a target substance which specifically binds to a member of specifically binding partner which was immobilized on the surface of the microreactor; and detecting formation of a bond between the member of specifically binding partner and the target substance.

Preferable embodiments in the aforementioned microreactor according to the present invention include the followings:

an embodiment wherein the specifically binding partner is bound to a part or entirety of the wall surface of the channel of the microreactor;

an embodiment wherein a plurality of the specifically binding partner are bound to different positions of the wall surface of the channel of the microreactor;

an embodiment wherein the specifically binding partner comprises a member which forms a biological specific bond;

an embodiment wherein the specifically binding partner is a combination of an antibody or antibody fragment and a ligand, a combination of an antibody or antibody fragment and an antigen, a combination of an antibody or antibody fragment and a hapten, or a combination of a receptor and a ligand;

an embodiment wherein the specifically binding partner is a combination of avidins and biotins;

an embodiment wherein the avidins are avidin, streptoavidin, or a modified compound thereof capable of forming a stable complex with biotin;

an embodiment wherein the biotins are biotin, biocytin, desthiobiotin, oxybiotin, or a derivative thereof capable of forming a stable complex with avidin;

an embodiment wherein the specifically binding partner is a combination of a nucleic acid and a nucleic acid, or a combination of a nucleic acid and a nucleic acid-binding substance;

an embodiment wherein the nucleic acid is a nucleotide derivative, a peptide nucleic acid, or LNA;

an embodiment wherein the nucleic acid-binding substance is a double-stranded DNA recognizing material;

an embodiment wherein the double-stranded DNA recognizing substance is a double-stranded DNA recognizing antibody;

an embodiment wherein the double-stranded DNA recognizing substance is a DNA transcription factor;

an embodiment wherein the double-stranded DNA recognizing substance is a protein having a Zn finger motif or a ring finger motif;

an embodiment wherein the double-stranded DNA recognizing material is a peptide nucleic acid;

an embodiment wherein A represents a residual group of a protein in the formula (3);

an embodiment wherein the wall surface of the channel inside the microreactor is glass, quartz, plastic, silicon resin, electrode surface, or sensor chip surface; and

an embodiment wherein the wall surface of the channel inside the microreactor is contacted with at least one of a member of specifically binding partner, and then a free reactive group present on the surface is subjected to blocking treatment with an aqueous solution of an amino acid, a peptide or a protein.

### DETAILED DESCRIPTION OF THE INVENTION

The mode of the present invention is described below in detail.

(1) Reactive Microreactor According to the Present Invention and Method for Producing it

The microreactor according to the present invention is a device having a channel with a 1 mm or smaller equivalent diameter. The microreactor can be produced, for example, on a solid substrate by micro processing technique, and also can be made by a molding method using a pattern on the solid substrate as a template.

The equivalent diameter in the present invention is a term also called a corresponding diameter, which is used in mechanical engineering field. If a cylindrical pipe equivalent to the pipe (channel in the present invention) having any sectional shape is assumed, the diameter of the equivalent cylindrical pipe corresponds to the equivalent diameter, and is defined as deq=4A/p using A: a sectional area of the pipe and p: a wetted perimeter length (circumferential length) of the pipe. In the case of the cylindrical pipe, this equivalent diameter corresponds to the diameter of the cylindrical pipe. The equivalent diameter is used for presuming fluidity or heat conducting characteristic of the pipe on the basis of data of the equivalent cylindrical pipe, and expresses a spatial scale (a representative length) of a phenomenon. The equivalent diameter is deq=4a²/4a=a in a squared pipe having a side a, deq=a/√3 in a triangular pipe having a side (a), and deq=2h in a flow between paralleled plates having a flow path height (h) (refer to Nippon Kikai Gakkai, Kikai Kougaku Jiten, 1997, published by Maruzen, K. K.).

The reaction device having such a flow path (channel) of a micro scale is commonly called “microreactor” and is being developed greatly in recent years (W. Ehrfeld. V. Hessel, H. Lowe, “Microreactor,” 1Ed. (2000) Wiley-VCH).

The channel in the present invention is formed on a solid substrate by micro processing technique. Examples of materials used include metal, silicon, Teflon (registered trade mark), glass, quartz, silicon resin, ceramics and plastics. When heat resistance, pressure resistance and solvent resistance are required, preferable materials include metal, silicon, Teflon (registered trade mark), glass and ceramics. Particularly preferred is metal. Metals include nickel, aluminium, silver, gold, platinum, tantalum, stainless steel, hastelloy (Ni—Fe based alloy) and titanium. Highly anticorrosive stainless steel, hastelloy or titanium is preferred.

Representative examples of micro processing techniques for producing the channel includes a LIGA method using X-ray lithography, high aspect ratio photolithography using EPON SU-8, a micro discharge processing method (μ-DEM), a high aspect ratio processing method for silicon by Deep RIE, a hot emboss processing method, a photo-fabrication method, a laser processing method, an ion beam processing method and a mechanical micro cutting processing method using a micro tool made of such a hard material as diamond. These techniques may be used alone or in combination. Preferable micro processing techniques are the LIGA method using X-ray lithography, the high aspect ratio photolithography using EPON SU-8, the micro discharge processing method (μ-DEM) and the mechanical cutting processing method.

The channel used for the present invention can be prepared also by using a pattern, which is made by applying a photoresist on a silicon wafer, as a template, and injecting a resin therein and solidify the resin (molding method). For the molding method, a silicon resin represented by PDMS (polydimethyl siloxane) or its derivatives can be used.

A temperature of the microreactor may be controlled by placing a whole device in a container of which temperature is controlled, or by thermal cycling by installing a metal resistor wire or a heater structure such as polysilicon in the device and using this for heating and natural cooling for cooling. For temperature sensing, in the metal resistor wire is prepared the same additional resistor wire as the heater to detect a temperature according to a change of the resistance value; in polysilicon is used a thermocouple to detect it. Also, heating and cooling may be carried out by contacting a Peltier element to the reactor. A method is selected depending on the use and the material of a main section of the reactor.

The wall surface of the channel of the microreactor can be coated with a polymer such as a polycationic compound (e.g., preferably poly-L-lysin, polyethylene imine, polyalkyl amine, etc., more preferably poly-L-lysin) having an amino group in the side chain in order to bind and fix a bifunctional reactive compound such as divinyl sulfon compound by a covalent bond. In this case, the reactive group which is introduced to the surface of the microreactor is an amino group. Alternatively, the wall surface of the channel of the microreactor can be contacted with a surface treatment agent such as a silane coupling agent, which has a reactive group which reacts to the surface of the microreactor and another reactive group such as an amino group.

In the case of coating treatment with the polycationic compound, an amino group or a mercapto group is introduced to the wall surface of the channel of the microreactor by the electrostatic bond of the polymer compound with the wall surface of the channel of the microreactor. On the contrary, in the case of treatment of the wall surface of the channel with the silane coupling agent, binding and fixing is carried out by the covalent bond to the wall surface of the channel of the microreactor, and therefore, an amino group or a mercapto group is stably present on the wall surface of the channel of the microreactor. Other than the amino group or the mercapto group, aldehyde group, epoxy group, carboxyl group or hydroxyl group can be preferably introduced.

γ-aminopropyl triethoxysilane, N-β (aminoethyl)-γ-aminopropyltrimethoxysilane or Nβ (aminoethyl)-γ-aminopropylmethyl dimethoxysilane is preferably used as the silane-coupling agent having an amino group. γ-aminopropyl triethoxysilane is particularly preferably used.

The treatment with the polycationic compound may be carried out in combination with the treatment with the silane-coupling agent. By this method, an electrostatic interaction can be promoted between a hydrophobic or low hydrophilic microreactor and the DNA fragment. On the wall surface of the channel of the microreactor, which was treated with the polycationic compound, may be additionally overlaid a layer made of a hydrophilic polymer having an electric charge or a layer made of a linker. By overlaying such the layer, irregular surface of the microreactor, which was treated with the polycationic compound, can be reduced. Depending on the type of the microreactor, the hydrophilic polymer can be contained therein, and the thus-trated microreactor can be used.

The microreactor having a reactive group is contacted with a bifunctional reactive compound such as a divinyl sulfon compound, and there active group is reacted with the bifunctional reactive compound to form a covalent bond. Thereby there active group moiety of the microreactor is elongated to form a reactive chain having a vinyl sulfonyl group or a reactive precursor group thereof in its end or around the end. Thus, the reactive microreactor according to the present invention is produced.

In the reactive microreactor according to the present invention, a ligated product of the vinyl sulfonyl group or its reactive precursor group thereof which is introduced to the wall surface of the channel and the linking group is represented by the following formulae (1):
-L-SO₂—X¹ (1)
wherein X¹ represents —CR¹═CR²R³ or —CHR¹—CR²R³Y (the reactive precursor group), R¹, R² and R³ independently represent an atom or a group selected from the group consisting of a hydrogen atom, a C1-6 alkyl group, a C6-20 aryl group and a C7-26 aralkyl group having a C1-6 alkyl chain.

Examples of C1-6 alkyl group include a methyl group, an ethyl group, an n-propyl group, an n-butyl group and an n-hexyl group, and methyl group is particularly preferable. Examples of C6-20 aryl group include a phenyl group and a naphthyl group. Each of R¹, R² and R³ is preferably a hydrogen atom.

Examples of C7-26 aralkyl group having a C1-6 alkyl chain include a combination of the aforementioned examples of the C1-6 alkyl groups and the aforementioned examples of the C6-20 aryl group.

Y represents a group which can be substituted by a nucleophilic reagent such as —OH, —OR⁰, —SH, NH₃, NH₂R⁰ (wherein R⁰ is a group such as alkyl group, excluding a hydrogen atom) or a group which is released as “HY” by a base. Examples thereof include a halogen atom, —OSO₂R²¹, —OCOR²², —OSO₃M, or a quaternary pyridinium group wherein R²¹ represents a C1-6 alkyl group, a C6-20 aryl group, and a C7-26 aralkyl group having C1-6 alkyl chain; R²² represents a C1-6 alkyl group or a C1-6 halogenated alkyl group; and M represents a hydrogen atom, an alkali metal atom or an ammonium group.

In the formula (1), L represents a bivalent or more linking group which binds the microreactor or a linking group which is bound to the microreactor, to the aforementioned —SO₂—X¹. L may be a single bond. Preferred examples of the bivalent linking group include a group selected from the group consisting of C1-6 alkylene group, C3-16alicyclic group, C6-20arylene group, C2-20 heterocyclic group having 1 to 3 heteroatoms selected from a group consisting of N, S and P, —O—, —S—, —SO—, —SO₂—, —SO₃—, —NR²³, —CO— or a combination thereof, or a group obtained by combination of two or more of the aforementioned groups. R²³ is preferably a hydrogen atom, a C1-15 alkyl group, a C6-20 aryl group or a C7-21 aralkyl group having a C1-6 alkyl group. More preferred examples are a hydrogen atom and a C1-15 alkyl group. Particularly preferred examples include a hydrogen atom, methyl group and ethyl group.

When L is a group obtained by combining two or more groups selected from the group consisting of —NR²³—, —SONR²³—, —CONR²³—, —NR²³COO— and —NR²³CONR²³—, a ring may be formed by binding of NR²³ therein.

The alkyl group of R²¹, the aryl group of R²¹ and the aralkyl group of R² may have a substituent. Examples of the substituent include an atom or a group selected from the group consisting of a hydroxyl group, a C1-6 alkoxy group, a C1-6 alkenyl group, a C2-7 carbamoyl group, a C1-6 alkyl group, a C7-16 aralkyl group, a C6-20 aryl group, a sulfamoyl group (or its sodium salt, potassium salt or the like), a sulfo group (or its sodium salt, potassium salt or the like), a carboxylic acid group (or its sodium salt, potassium salt or the like), a halogen atom, a C1-6 alkenylene group, a C6-20 arylene group, a sulfonyl group, and combination thereof.

Preferred examples of “—X¹” is shown below. In addition, the example which can be used as “-L-SO₂—X¹” is shown below.

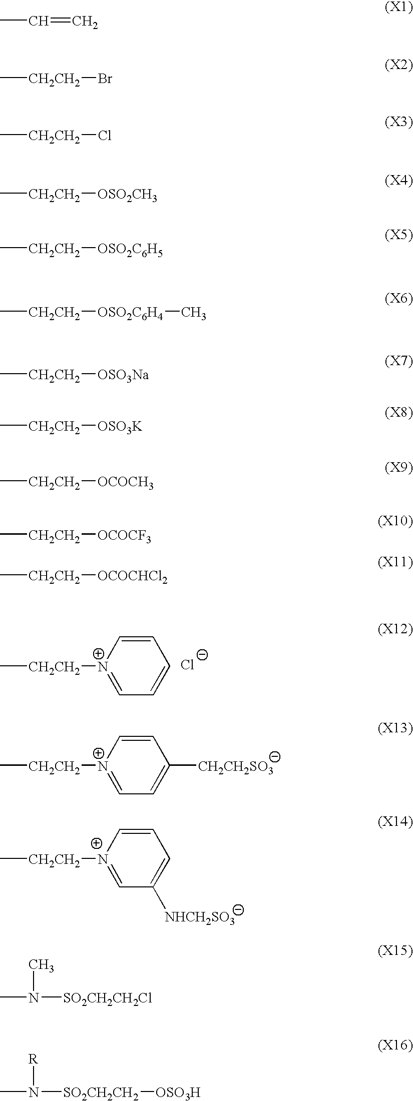

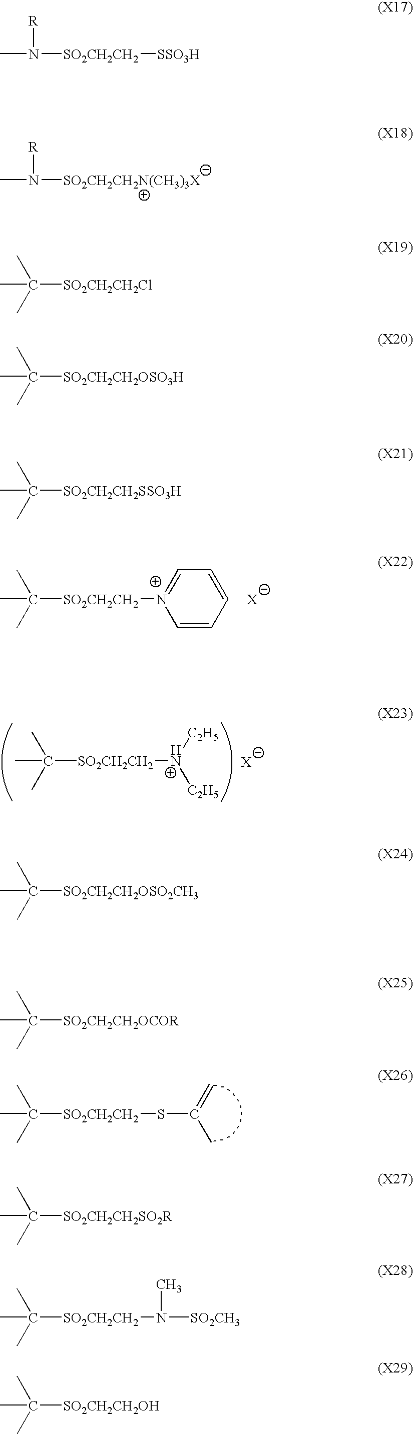

“—X¹” is preferably (X1), (X2), (X3), (X4,) (X7), (X8), (X13) or (X14), and more preferably (X1) or (X2), in the aforementioned examples. Particularly preferred example is the vinyl group represented by (X1).

The preferable examples of L are shown below. “a” represents an integer of 1 to 6, and preferably 1 or 2, particularly preferably 1. “b” is an integer of 0 to 6 and preferably 2 or 3.

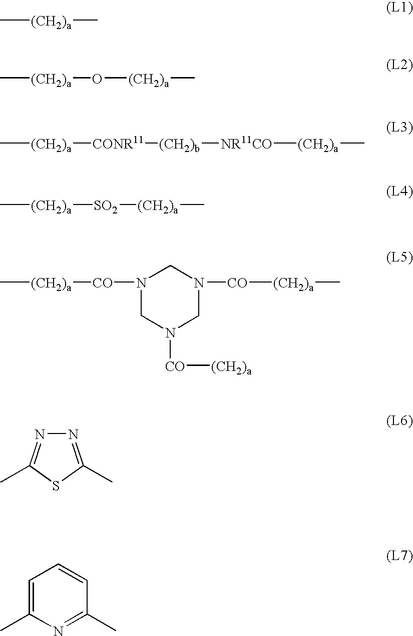

As for L, as well as the bivalent linking group as described above, a group obtained by substituting the hydrogen atom of the alkylene group of the above formula with —SO₂CH═CH₂ is also preferable.

As for a bivalent reactive compound used for obtaining the microreactor where the vinyl sulfonyl group of the aforementioned formula (1) or the reactive precursor group is fixed by a covalent bond, a disulfon compound represented by the formula (2) can be advantageously used.
X¹—SO₂-L²-SO₂—X² (2)

In the formula (2), X¹ and X² independently represent —CR¹═CR²R³ or —CHR¹—CR²R³Y; R¹, R² and R³ independently represent an atom or a group selected from the group consisting of a hydrogen atom, a C1-6 alkyl group, a C6-20 aryl group and a C7-26 aralkyl group having a C1-6 alkyl chain; Y represents a group which can be substituted by a nucleophilic reagent or a group which is released as “HY” by a base; and L² represents a linking group. The examples of individual groups in the formula (2) are the same as those of the examples of individual groups in the formula (1).

The disulfon compound represented by the formula (2) is contacted with the aforementioned microreactor under aqueous atmosphere to easily produce the reactive microreactor according to the present invention.

The typical examples of the disulfon compound preferably used in the present invention are shown below. The disulfon compound may be used by combining two or more types of the compound.

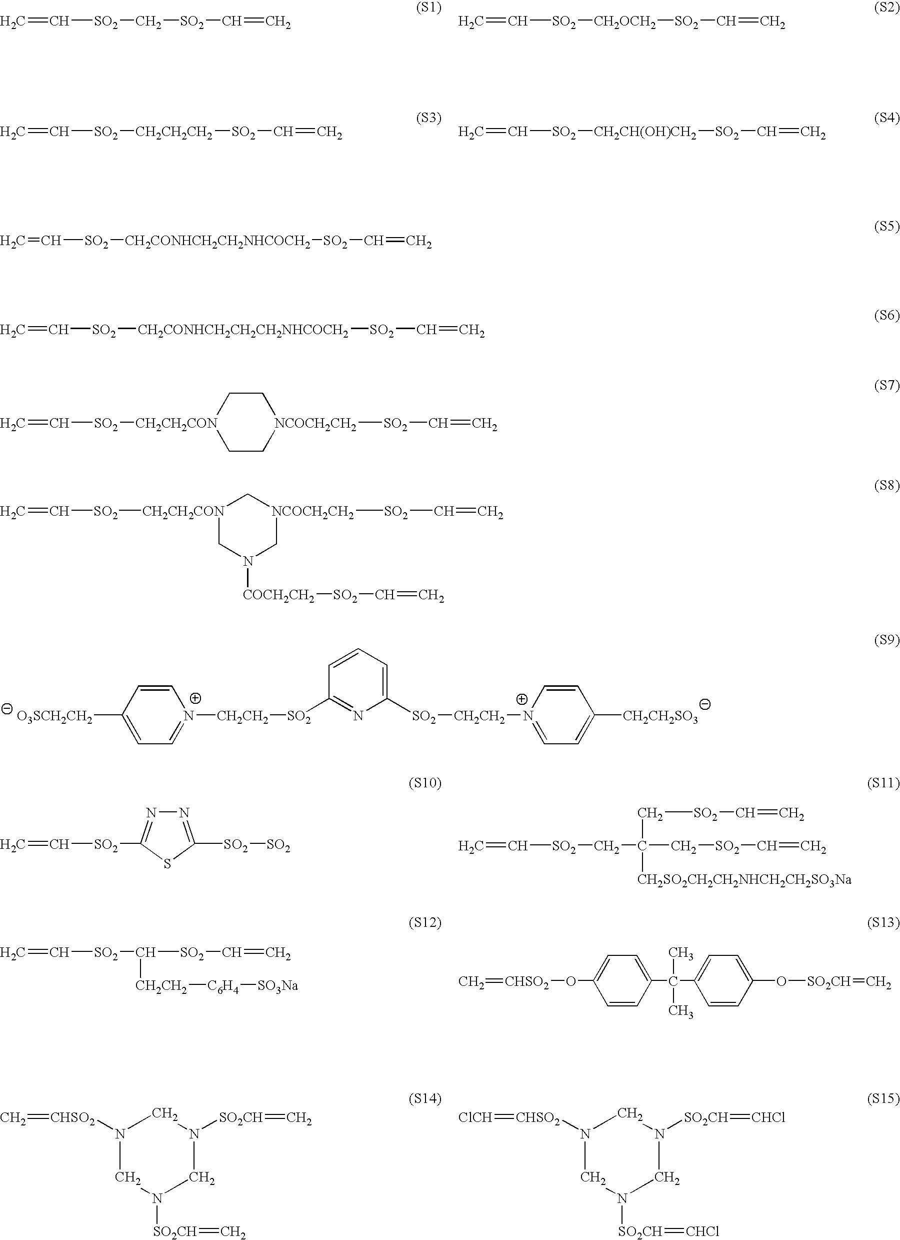

The typical example of the disulfon compounds represented by the formula (2) is 1,2-bis(vinylsulfonylacetamide)ethane which corresponds to S1 of the above.

The detail of the method of synthesizing the disulfon compound used in the present invention is described in various publications such as Japanese Patent Publication No. 1972-2429 and No. 1975-35807, Japanese Patent Laid-open Publication No. 1974-24435, No. 1978-41551 and No. 1984-18944, and the like.

(2) Biological Material-Bound Microreactor According to the Present Invention and Method for Producing it

In the biological material-bound microreactor according to the present invention, a group of the following formula (3) having a residual group of a member of specifically binding partner is bound to the wall surface of a channel inside the microreactor:
-L-SO₂—X-A (3)
wherein L represents a linking group which bind —SO₂—X-A to the wall surface of the channel inside the microreactor; X represents —CR¹¹(R¹²)—CR¹³(R¹⁴)—; R¹¹, R¹², R¹³ and R¹⁴ independently represent a hydrogen atom, a C1-6 alkyl group, a C6-20 aryl group or a C7-26 aralkyl group having a C1-6 alkyl chain; A represents a residual group of the member of the specifically binding partner.

The specifically binding partner in the present invention means a binding partner which forms a biologically specific bond, and the types thereof are not especially limited. Examples thereof include the combination of those having an antigenic determinant, such as an antibody or antibody fragment/a ligand, an antibody or antibody fragment/an antigen, an antibody or antibody fragment/a hapten, and the combination of a receptor/a ligand, avidins/biotins, a nucleic acid/a nucleic acid, and a nucleic acid/a nucleic acid-binding protein. In the case where the DNA fixed solid support which is produced according to the present invention is used as the DNA chip, a specific binding strength is achieved to allow a repeated use accurately for subsequent hybridization operation.

Examples of biotins include biotin, biocytin, desthiobiotin, oxybiotin, and the derivative thereof capable of forming a stable complex with avidin. The phrase “capable of forming a stable complex with avidin” means capability of forming a complex having a dissociation constant similar to the dissociation constant (10⁻¹⁵ M) of a biotin-avidin complex. On the other hand, examples of avidins include avidin, streptoavidin, and the modified compound thereof capable of forming a stable complex with biotin. A sense of the “stable complex” herein is the same as the definition for the biotins as described above. In addition, the modified compound means a modified compound or the fragment of naturally derived avidins or streptoavidin or a recombinant thereof.

The nucleic acid is not especially limited, and a nucleotide derivative or its analogue is exemplified. Typical examples include oligonucleotide, polynucleotide, and peptide nucleic acid. These nucleotide derivatives or analogues thereof may be those naturally derived (such as DNA, DNA fragment, RNA or RNA fragment) or a synthetic compound. Moreover, the nucleotide derivatives or analogues thereof include various analogue compounds such as a compound having a crosslinking group in its sugar unit portion, called as LNA (described in J. Am. Chem. Soc. 1998, 120, 13252-13253).

A nucleic acid-binding substance is not especially limited, and may be a double-stranded DNA recognizing substance. The double-stranded DNA recognizing substance means a substance which recognizes the double-stranded DNA and bind specifically thereto. Examples of the double-stranded DNA recognizing substance include a DNA transcription factor, a mismatch repair protein, a double-stranded DNA recognizing antibody and a peptide nucleic acid. The double-stranded recognizing substance includes those having the Zn finger motif or the ring finger motif.

The DNA transcription factor is a substance which binds to a promoter region on a gene and regulates transcription from DNA to mRNA (Tamura Takaaki: Transcription Factor (Youdosya, 1995)). Thus, it has been known that the transcription factor is bound specifically to the double-stranded DNA having a specific sequence.

Among many existing transcription factors, a Zinc finger protein, i.e., a group of the transcription factors having the Zinc finger motif or the Ring Finger motif, show a very high occurrence ratio in eukaryotes and probably encodes 1% of the genome. Pabo et al. have analyzed a tertiary structure of the Zinc finger motif and have solved a mechanism of binding with the DNA (Science 252: 809. (1991)). In addition, Choo et al. have successfully prepared a group of Zinc finger proteins which are bound to the specific sequence and are absent in nature world, by gene recombination method (Nature 372: 642. 1994; PNAS 91: 11163. 1994). Furthermore, a research team of Scripps Research Institute has successfully prepared a group of new Zinc finger proteins by phage display (PNAS 95: 2812.1998, 96: 2758. 1999). Hence, the group of DNA transcription factors, typical example of which is the Zinc finger protein, has inherently a property of binding to the double-stranded DNA. In addition, according to recent studies, it has become possible to prepare a recombinant which recognizes an arbitrary DNA sequence. By fixing such the protein, the double-stranded DNA can be efficiently captured on the support.

In addition, the nucleic acid-binding substance includes a helix loop helix protein and a protein having an Ets domain.

In the case where the member of the specifically binding partner to be fixed on the wall surface of the channel inside the microreactor is a protein, the proteins form a covalent bond with a reactive group through the sulfonyl group by using their internally existing amino group or mercapto group, or the reactive group, that is prepared by introducing an amino group, imino group, hydrazino group, carbamoyl group, hydrazinocarbonyl group, mercapto group, or carboxyimido group to a protein.

The wall surface of the channel inside the microreactor, on which the member (e.g., antibody, avidins, nucleic acid-binding substance) of the specifically binding partner which is the protein as described above is fixed, may be contacted with another member (e.g., ligand, biotins, nucleic acid) of the binding partner which specifically reacts with the binding partner under an aqueous medium, and thus the binding partner can be fixed. It is preferable that another member (e.g., ligand, biotins, nucleic acid) of the specifically reactive binding partner to be fixed, is labeled with detectable label (e.g., fluorescent label or enzyme label) in such away that the fixation can be detected from outside.

When a nucleic acid is fixed to the wall surface of the channel inside the microreactor, typical examples of a nucleotide derivative or its analogues include oligonucleotide, polynucleotide, peptide nucleic acid and LNA. Examples of the nucleotide derivative or its analogue which can be used include those having a reactive group that reacts to the vinylsulfonyl group or its reactive precursor group to form a covalent bond, such as an amino group, imino group, hydrazino group, carbamoyl group, hydrazinocarbonyl group, carboxyimido group or mercapto group, at one end of the molecule or around the end.

The wall surface of the channel inside the microreactor, on which a nucleotide derivative or its analogue has been fixed, can be contacted with an oligonucleotide or polynucleotide (DNA or its fragment or RNA or its fragment) which is complementary to the fixed nucleotide derivative or its analogue in the presence of the aqueous medium, so as to cause hybridization. Thus, the complementary oligonucleotide or polynucleotide can be fixed. It is preferable that the complementary oligonucleotide or polynucleotide to be fixed is labeled with a detectable label (e.g., fluorescent label) in such a way that the fixation can be detected from outside.

In the present invention, the member (a residual group represented by A in the following formula (3)) of the specifically binding partner of the biological material-bound microreactor is bound to the wall surface of the channel of the microreactor by the covalent bond via the sulfonyl group as shown in the following formula (3):
-L-SO₂—X-A (3)
wherein L represents a linking group which bind —SO₂—X-A to the wall surface of the channel inside the microreactor; X represents —CR¹¹(R¹²)—CR¹³(R¹⁴)—; R¹¹, R¹²,R¹³ and R¹⁴ independently represent a hydrogen atom, a C1-6 alkyl group, a C6-20 aryl group or a C7-26 aralkyl group having a C1-6 alkyl chain; A represents a residual group of the member of the specifically binding partner.

In the formula (3), L represents a bivalent or more linking group which bind —SO₂—X-A to the wall surface of the channel inside the microreactor. Examples of -L- are same as those of the example of -L- in the formula (1).

In the formula (3), examples of the C1-6 alkyl group include a methyl group, ethyl, group, n-propyl group, n-butyl group and n-hexyl group, and methyl group is particularly preferable. Examples of the C6-20 aryl group include a phenyl group and naphthyl group. Each of R¹, R² and R³ is preferably a hydrogen atom.

Examples of the C7-26 aralkyl group having a C1-6 alkyl chain include the combination of the aforementioned examples of the C1-6 alkyl groups and the aforementioned examples of the C6-20 aryl group.

The biological material-bound microreactor according to the present invention can be produced by contacting the wall surface of the channel inside the microreactor having a vinylsulfonyl group represented by the aforementioned formula (1) or its reactive precursor group, with at least one of a member of specifically binding partner having a reactive group which forms a covalent bond by reacting with the vinylsulfonyl group or its reactive precursor group.

The vinylsulfonyl group or its reactive precursor group, which is bound to the wall surface of the channel of the microreactor of the present invention via the covalent bond, has a high resistance against hydrolysis and thus, can be easily and stably kept. In addition, the vinylsulfonyl group or its reactive precursor group reacts quickly to the member of the specifically binding partner which previously has a reactive group such as an amino group or to which a reactive group such as an amino group has been introduced, so as to form a stable covalent bond.

To one end of the nucleotide derivative or its analogue such as oligonucleotide or DNA fragment, a reactive group which forms a covalent bond by reaction with the aforementioned vinylsulfonyl group or its reactive precursor group, is introduced. Such reactive group is preferably an amino group, imino group, hydrazino group, carbamoyl group, hydrazinocarbonyl group, carboxyimido group or mercapto group, and particularly preferably amino group. To the oligonucleotide or the DNA fragment is bound a reactive group usually via a crosslinker. As the crosslinker, for example, an alkylene group or an N-alkyl-amino-alkylene group is used. Preferably, a hexylene group or an N-methylamino-hexylene group is used. Particularly preferably, hexylene group is used. Since the peptide nucleic acid (PNA) has an amino group, usually, it is not necessary to introduce another reactive group.

Similarly, the protein has an amino group ormercapto group and hence, usually, it is not necessary to introduce another reactive group. However, a tertiary structure of the protein is greatly involved in its function. Therefore, when an activity of the protein is reduced, it is preferable to introduce a reactive group at a specific position which does not influence the activity.

For the same purpose, it is also preferable to place the microreactor, while keeping a contact the microreactor with the nucleotide derivative, its analogue or the protein, in an environment of a humidity of 90% or higher and a temperature ranging from 25 to 50° C. (for proteins, up to 37° C.)

A fixed amount (number) of a biological material (i.e., the member of specifically binding partner) such as a protein, a nucleotide derivative or its analogue is within the range from 1 to 10⁷ types/cm² for a surface of the wall surface of the channel inside the microreactor.

(3) Method for Detecting a Target Substance Contained in a Sample

The invention further relates to a method for detecting a target substance, which comprises steps of: (a) contacting the biological material-bound microreactor wherein a group of the aforementioned formula (3) having a residual group of a member of specifically binding partner is bound to the wall surface of a channel inside the microreactor, with a sample containing a target substance (i.e., a target substance which is another member of the aforementioned specifically binding partner) which specifically binds to a member of specifically binding partner which was immobilized on the surface of the microreactor; and (b) detecting formation of a bond between the member of specifically binding partner and the target substance.

The type of “the test sample containing the target substance” is not especially limited. For example, blood such as peripheral vein blood, a tissue cell such as a leukocyte, plasma, urine, feces, semen, saliva, cultured cell and various organ cells, and other any samples containing a nucleic acid, can be used in the present invention. As the sample, a sample such as the tissue cell as described above may be used intactly. Preferably, the nucleic acid and ligand are isolated by breaking the cell in the sample, and are used as a sample. The cell in the sample is broken by an ordinary method and for example, physical actions such as shaking or ultrasonication can be applied from outside. Alternatively, by using a nucleic acid-extracting solution (for example, a solution containing a surfactant such as SDS, Triton X or Tween 20, or saponin, EDTA, protease, or the like), the nucleic acid may be released from the cell. For extracting the nucleic acid using the nucleic acid-extracting solution, the reaction may be promoted by incubating the sample at a temperature of 37° C. or higher.

A protein chip which is a representative embodiment of the present invention is used for protein interaction analysis, protein expression analysis and drug development studies. Moreover, in the case of a nucleic acid-bound protein, the chip may be used for mutation analysis and polymorphism analysis depending on the recognized nucleic acid sequence.

A principle of detection is based on the reaction to the ligand or the nucleic acid that was labeled. As a labeling method, an RI method and a non-RI method (fluorescence method, biotin method and chemoluminescence method) are known, and the labeling method is not especially limited. For example, in the case of the fluorescence method, a fluorescent substance which is used for fluorescence labeling may be any one of those which can be bound to a base portion of a nucleic acid or an amino acid residue of a protein. For example, a cyanine dye (e.g., Cy3, Cy5, etc of commercially available Cy Dye TM series), a rhodamine 6G reagent, N-acetoxy-N2-acetylamino fluorene (AAF) or AAIF (an iodine derivative of AAF) may be used.

It is preferable to detect directly the target nucleic acid in the test sample without amplification by PCR. However, detection may be carried out after previous amplification. The target nucleic acid or its amplified product may be easily detected by previous labeling. The nucleic acid is often labeled by using enzyme (Reverse Transcriptase, DNA polymerase, RNA polymerase, terminal deoxytransferase, and the like). The target substance may be directly bound by a chemical reaction. These labeling methods have been described in books as publicly known techniques (Nomura Sintarou, Non-isotope Experimental Protocol 1, Syuzyunsya, 1994; Non-isotope Experimental Protocol 2, Syuzyunsya, 1998; Muramatu Masaaki, DNAMicroarray and Newest Labeling Substance for PCR Method, Syuzyunsya, 2000). The labeling substance is preferably a substance capable of generating a detectable signal. When the labeling substance is a substance such as an enzyme and a catalyst, which can amplify the signal, sensitivity of detection of the DNA is greatly increased.

However, the labeling procedure as described above is generally complicated. More preferable detection method is exemplified by a method for measuring the nucleic acid in the sample without previous labeling. For this purpose, for example, a DNA intercalating agent which recognizes the double-stranded DNA, i.e., a DNA intercalator, can be used. By the use of the DNA intercalator, not only the detection procedure becomes easy, but also the detection sensitivity becomes higher. For example, for the detection of 1000 bp DNA, the labeling method introduces only some molecules of the labeling substance at most, but when the intercalator is used, 100 or more molecules of the labeling substance can be introduced.

The DNA intercalator may be a substance capable of forming a detectable signal by itself. Alternatively, a signal-forming substance may be bound to a side chain of a DNA intercalator, or to the DNA intercalator via a specific binding pair such as biotin-avidin, antigen-antibody or hapten-antibody. Preferred examples of the detectable signal in the present invention include signals which can be detected by fluorescence detection, luminescence detection, chemoluminescence detection, bioluminescence detection, electrochemical luminescence detection, radioactivity detection, electrochemical detection or calorimetric detection, but are not limited thereto.

When the ligand is a target, those prepared by reacting an internally existing amino acid to a succinimide of cyanine dye (e.g., Cy3, Cy5, etc of commercially available Cy Dye TM series), a rhodamine 6G reagent, N-acetoxy-N2-acetylamino fluorene (AAF) or AAIF (the iodine derivative of AAF) can be used.

The present invention will be described more specifically with reference to the following Examples. However, the present invention is not limited by the Examples.

### EXAMPLES

(1) Preparation of a Quartz Microreactor

A channel pattern with a 100 μm width was formed on the surface of a quartz glass by photolithography using Au/Cr. The pattern was used as a mask, and was subjected to etching treatment with hydrofluoric acid to form a groove. To this product was bonded a quartz glass by hydrofluoric acid binding method to prepare a quartz microreactor having a groove as the channel.

(2) Preparation of the Microreactor (1) wherein the Vinylsulfonyl Group has Been Introduced to the Wall Surface of the Channel

A solution of aminopropyl ethoxy silane (Shin-Etsu Chemical Co., LTD) (2% by weight) in ethanol, was injected into the channel of the microreactor prepared in the above (1), and the microreactor was left stand for 10 minutes. Ethanol was injected into the channel for washing, followed by drying at 110° C. for 10 minutes to prepare a silane compound-coated slide. Into the channel of this silane compound-coated slide was injected a phosphate buffer solution (pH 8.5) of 1,2-bis(vinylsulfonylacetamide)ethane (5% by weight), and the slide was left stand for 1 hour. Subsequently, the slide was washed with acetonitrile, and dried at a reduced pressure for 1 hour to prepare a microreactor in which a vinylsulfonyl group was introduced to its wall surface.

(3) Preparation of Flat Quartz Glass in which a Vinylsulfonyl Group was Introduced to its Surface (a Control)

According to the same method as in Example (2), flat quartz (2) in which a vinylsulfonyl group was introduced to its surface, was prepared.

(4) Detection of a Ligand Using an Antibody-Fixed Slide Glass

(4-1) Fixation of an Antibody

Goat Anti Human IgG H+L Specific (Jackson ImmunoResearch made) was diluted (100 ng/μL) with PBS, and 0.5 μL of it was injected into the channel of the microreactor (1) prepared in Example (2) as described above, in which a vinylsulfonyl group was introduced to its wall surface. After being left stand for 3 hours, 3% casein/0.05% Tween 20-PBS (PBS-T) was injected, and then the microreactor was left stand for 1 hour, followed by washing with PBS to obtain an antibody-fixed microreactor.

Similarly, 2 μL of the diluted solution of Goat Anti Human IgG H+L Specific (Jackson ImmunoResearch made) was spotted on the preparation of flat quartz glass (the control), and the glass was left stand for 3 hours. Then, 3% casein/0.05% Tween 20-PBS (PBS-T) was injected and the glass was left stand for 1 hour for blocking.

(4-2) Detection of Human IgG

Into the antibody-fixed microreactor (1) prepared in the above (4-1) was injected 0.5 μL of Human IgG (Jackson ImmunoResearch made), and the microreactor was left stand for 15 minutes. Then, PBS was injected therein for washing. Next, 0.1 μL of Goat Anti Human IgG Fc Specific-HRP (Jackson ImmunoResearch made) which was 1000-fold diluted with a 0.1% casein-containing PBS, was injected, and the microreactor was left stand for 15 minutes. Then, PBS was injected for washing. In addition, 0.5 μL of an ECL luminescent substrate (Amersham made) was injected, and then luminescence intensity was measured by using LAS1000 (Fuji Photo Film Co., LTD. made).

In the same way, an antibody-fixed flat quartz (2) was immersed in 2 ml of a PBS solution of Human IgG, and the flat quartz was left stand for 15 minutes and washed with PBS. The flat quartz (2) was immersed for 15 minutes in 2 ml of Goat Anti Human IgG Fc Specific-HRP (Jackson ImmunoResearch made) which was 1000-fold diluted with a 0.1% casein-containing PBS. In addition, the flat quartz (2) was immersed in 1 mL of an ECL luminescent substrate (Amersham made) and then, luminescence intensity was measured by using LAS1000 (Fuji Photo Film Col, LTD. made). The results were shown in the following Table 1.

TABLE 1

| | | Microreactor (1) | Flat quartz (2) |
|---|---|---|---|
| | IgG | of the invention | of Control |
| | concentration | luminescence | luminescence |
| No. | (ng/ml) | intensity | intensity |
| 1 | 100 | 15600 | 8500 |
| 2 | 10 | 8250 | 6300 |
| 3 | 1 | 5300 | 4200 |
| 4 | 0 | 3800 | 3900 |

From the results, it is clear that an antibody is effectively bound in the microreactor according to the present invention. Also it is clear that in comparison with a plate reactor as the control, the microreactor according to the present invention requires a less amount of a reagent and achieves a higher S/N ratio.

### EFFECT OF THE INVENTION

The invention provides a reactive microreactor which achieves quick and stable binding and fixation; a biological material-bound microreactor prepared by binding and fixing at least one of a member of specifically binding partner to the wall surface of the channel inside the reactive microreactor; a method for producing the same: and a method for using the same. The microreactor according to the present invention has advantages that it requires a less amount of a reagent and achieves a higher S/N ratio.

1. (canceled)

2. A method for producing a reactive microreactor wherein a group of the following formula (1) which can bind to a member of specifically binding partner, is bound to a part or entirety of a wall surface of a channel:
-L-SO₂—X¹ (1)
wherein X¹ represents —CR¹═CR²R³ or —CHR¹—CR²R³Y; R¹, R² and R³ independently represent an atom or a group selected from the group consisting of a hydrogen atom, a C1-6 alkyl group a C6-20 aryl group and a C7-20 aralkyl group having a C1-6 alkyl chain; Y represents a group which can be substitutable by a nucleophilic reagent or a group which is released as “HY” by a base: and L represents a linking group;
the method comprising contacting a microreactor where a reactive group is introduced on the surface with a disulfon compound of the following formula (2):
X¹—SO₂-L²-SO₂—X² (2)
wherein X¹ and X² independently represent —CR¹═CR²R³ or —CHR¹—CR²R³Y; R¹, R² and R³ independently represent an atom or a group selected from the group consisting of a hydrogen atom, a C1-6 alkyl group, a C6-20 aryl group and a C7-26 aralkyl group having a C1-6 alkyl chain; Y represents a group which can be substituted by a nucleophilic reagent or a group which is released as “HY” by a base; and L² represents a linking group.

3-15. (canceled)

16. A method for producing a biological material-bound microreactor wherein a group of the following formula (3) having a residual group of a member of specifically binding partner is bound to a part or entirety of a wall surface of a channel
-L-SO₂—X-A (3)
wherein L represents a linking group which binds —SO₂—X-A to the wall surface of the channel inside the microreactor; X represents —CR¹¹(R¹²)—CR¹³(R¹⁴)—; R¹¹, R¹², R¹³ and R¹⁴ independently represent a hydrogen atom, a C1-6 alkyl group, a C6-20 aryl group or a C7-26 aralkyl group having a C1-6 alkyl chain; A represents a residual group of the member of the specifically binding partner;
the method comprising a step of contacting a reactive microreactor of wherein a group of the following formula (1) which can bind to a member of specifically binding partner, is bound to a part or entirety of a wall surface of a channel:
-L-SO₂—X¹ (1)
wherein X¹ represents —CR¹═CR²R³ or —CHR¹—CR²R³Y R¹, R² and R³ independently represent an atom or a group selected from the group consisting of a hydrogen atom, a C1-6 alkyl group, a C6-20 aryl group and a C7-20 aralkyl group having a C1-6 alkyl chain; Y represents a group which can be substitutable by a nucleophilic reagent or a group which is released as “HY” by a base; and L represents a linking group, with at least one of a member of specifically binding partner having a reactive group which reacts with a group of the aforementioned formula (1) and forms a covalent bond.

17. The method according to claim 16, wherein the wall surface of the channel inside the microreactor is contacted with at least one of a member of specifically binding partner, and then a free reactive group present on the surface is subjected to blocking treatment with an aqueous solution of an amino acid, a peptide or a protein.

18. (canceled)
